(19) United States
(12) Patent Application Publication
(10) Pub. No.: US 2004/0191114 A1
(43) Pub. Date: Sep. 30, 2004

(54) Process for sterilizing objects

(75) Inventors: Robert Frost, Landshut (DE); Gernot Keil, Munchen (DE); Peter Georg Scheubert, Grosshelfendorf (DE)

Correspondence Address: CROWELL & MORING LLP, INTELLECTUAL PROPERTY GROUP, P.O. BOX 14300, WASHINGTON, DC, 20044-4300, US

(73) Assignee: Rudiger Haaga GmbH

(21) Appl. No.: 10/806,292

(22) Filed: Mar. 23, 2004

(30) Foreign Application Priority Data
Mar. 27, 2003 (DE) 10314687.3

(51) Int. Cl. A61L 2/20

(52) U.S. Cl. 422/27

(57) ABSTRACT

Described is a process for sterilizing objects in a sterilization chamber in which a vacuum prevails, in which sterilization chamber a vapour mix consisting of water steam and hydrogen peroxide steam is fed without the use of carrier gas. The vapour mix is deposited in the form of a condensate layer abruptly on the surfaces of the objects to be sterilized and on the surfaces of the sterilization chamber. The condenste layer is subsequently sucked out by means of further evacuation of the sterilization chamber. In accordance with the present invention, the surfaces of the objects to be sterilized and/or the surfaces of the sterilization chamber are pre-heated to a pre-determined temperature.

### BACKGROUND AND SUMMARY OF THE INVENTION

This application claims the priority of German application 10314687.3, filed Mar. 27, 2003, the disclosure of which is expressly incorporated by reference herein.

The present invention relates to a process for sterilizing objects in a sterilization chamber in which a vacuum prevails, in which sterilization chamber a vapor mix consisting of water steam and hydrogen peroxide steam is fed without the use of carrier gas, the said vapor mix being deposited in the form of a condensate layer abruptly on the surfaces of the objects to be sterilized and on the surfaces of the sterilization chamber, where after the condensate layer is subsequently sucked out by means of further evacuation of the sterilization chamber.

A process of this type is prior art in German published patent application 101 14 758. In the known process, the condensate layer is abruptly deposited in a time spanning a few tenths of a second to a maximum of a few seconds, namely by means of undercooling and the subsequent over saturation of the steam phase. This is achieved by a high drop in pressure between the evaporator and the sterilization chamber due to the pre-evacuation of the sterilization chamber. Because of the abrupt application of the condensate layer, the hydrogen peroxide is activated, in that the entire evaporation heat is abruptly released and the resulting condensate heated, as it cannot be abruptly dissipated by means of thermal conduction. By means of this abrupt activating of the formed condensate layer, a short acting time for the highest rate of destruction of bacteria is achieved. The applicant presumes that the heating of the condensate results in dissociation, which then effects the damage to the microorganisms.

The above attempted explanation for the high effectiveness of the sterilization process cannot be found to this degree of detail in prior art, but can, however, be derived from the content of the disclosure. The present invention is based on a sterilization process of this type.

It has been shown that in the case of the known sterilization process the temperature ratios play a deciding role in its economic viability. It has also been shown that the amount of residue of hydrogen peroxide which remains after the condensate has been removed from the surfaces varies with the differences in temperature.

The applicant presumes that an adsorbent hydrogen peroxide water layer exists on the surfaces of the objects to be sterilized and on the sterilization chamber. The reason for this may be that the surfaces are not homogenous, which results in local electrical charging centers which are positive at one place while being negative at another. Electrical dipoles could then attach themselves to the electrical surface charges, which dipoles brush over the surfaces and in proportion to the distribution of the loads, arrange themselves thereon. Because of this above mentioned adsorption process, the particles attach themselves to each other due to the opposing electrical charges. In the present case, water and hydrogen peroxide molecules from a steam phase are involved, which have very strong dipoles and which adsorb quickly and effectively on all available surfaces.

During evacuation, subsequent to the condensate layer being applied, only those particles which are mobile can be sucked out. Adsorbed particles on the other hand, in the present case the strong dipoles of water and hydrogen peroxide, are deposited partly on the sterilized surfaces due to the electrical interaction. They are only then released from the surfaces when by chance they receive a hefty knock due to the thermal motion which, in that moment, prevails over the forces of adsorption.

It is an object of the present invention to make the process of the above mentioned type even more effective.

This object has been achieved in that the surfaces of the objects to be sterilized and/or the sterilization chamber are pre-heated up to a pre-determined temperature.

By increasing the temperature on the sterilized surfaces, the thermal movements of the surface atoms become more active. The increase in temperature should, of course, not be so strong that the abrupt condensation process, which has just previously taken place, is impaired. It has been shown that a quite moderate increase in temperature of the surfaces to be sterilized is sufficient in order to retain an increased desorption rate and, without increasing the evacuation time, to also retain a reduced residue of hydrogen peroxide. Pre-heating the surfaces to a temperature between 30° C. and 54° C., preferably between 34° C. and 46° C., appears to be advantageous.

The process according to the present invention not only results in an increased temperature of the existing overall system comprising the sterilization chamber and the objects to be sterilized, but rather increases also the steam pressure of the condensate layer. Thus the mass to be sucked out is transported at an increased pressure, which reduces the duration of the pumping-out process considerably. Because of the reduced duration of the pumping-out process during the removal of the condensate layer, evacuation can take place at a lower pressure, which results additionally in a reduction of the hydrogen peroxide residue. The pre-heating of the sterilization chamber results overall in a significant acceleration of the process.

It should be noted at this point that the present invention in no way excludes measures to achieve even greater economic feasilibility by means of additonal flooding of the sterilization chamber with hot air or by means of covering of the objects with hot air at a point downstream.

The present invention can be realized by applying different measures.

It can, for example, be provided that the surfaces to be sterilized are pre-heated by means of installed heating elements. It appears to be more advantageous, however, when the surfaces are pre-heated by means of warm air. This need not only apply to the sterilization chamber, but can also include the feeding devices for guiding the objects into the sterilization chamber. What is important is to achieve a temperature in the inside of the sterilization chamber that is as homogenous as possible, and to avoid cold areas.

In an embodiment of the present invention, a regulated, sterile air current can serve as warm air. There are sterilization installations in which a sterile air current, directed against the intake of the objects, is present in any case. When this sterile air current is heated by regulation, then an increased temperature is effected without the application of any further measures in the entire section, including if need be the feeding device of the objects into the sterilization chamber.

In a further embodiment of the present invention, the objects can be pre-heated before they are fed into the sterilization chamber. The heating of the sterilization chamber can then, in certain circumstances, be superfluous. A suitable pre-heating device could be installed directly upstream of the sterilization chamber, so that the objects to be sterilized are automatically heated during their transport into the chamber.

The present invention in its broadest interpretation is not dependent on any particular embodiment of the sterilization installation. The present invention can thus be advantageously applied in the case of so called rotatary machines as well as in the case of linear, cyclical machines. Furthermore, in the case of the present invention, it is irrelevant whether the respective sterilization chamber can take up only one object or a plurality of objects.

Other objects, advantages and novel features of the present invention will become apparent from the following detailed description of the invention when considered in conjunction with the accompanying drawings.

### BRIEF DESCRIPTION OF THE DRAWINGS

FIG. 1 is an installation for a sterilization process according to the present invention comprising a device for pre-heating the surfaces of the objects to be sterilized and of the sterilization chamber,

FIG. 2 is the application of the process according to the present invention in connection with an additional pre-heating of the objects to be sterilized before they are fed into a sterilization chamber.

### DETAILED DESCRIPTION OF THE DRAWINGS

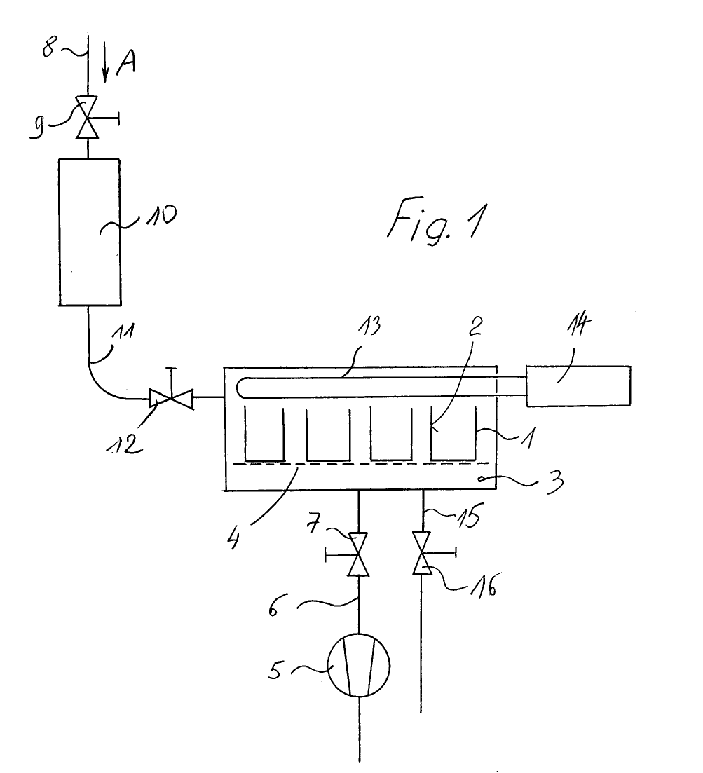

The objects 1 to be sterilized involve, for example, containers for drinks, whose surfaces 2 must be made free of microorganisms. In FIG. 1, four such objects 1 are shown in an evacuable sterilization chamber 3. A transport device 4, for example a perforated transport belt and denoted only by a broken line, can serve here as a supporting surface.

The sterilization chamber 3 is connected via a suction conduit 6 and a valve 7 to a vacuum pump 5. The sterilization chamber 3 can thus be evacuated to the pressures required for the process of the invention.

An evaporator 10 is arranged upstream of the sterilization chamber 3, to which evaporator 10 a fluid mix of water and hydrogen peroxide is fed, by means of a feed device 8, in delivery direction A via a valve 9. The resulting vapour mix is then guided without the aid of carrier gas via a conduit 11 and a valve 12 at a pre-determined time into the sterilization chamber 3.

In the installation according to FIG. 1, a vapour mix consisting of overheated water steam and overheated hydrogen peroxide steam is generated, whereby the construction of the evaporator 10 is basically irrelevant. The watery solution of hydrogen peroxide fed to the evaporator 10 has a concentration of, for example, 30% to 50%.

After the evacuation of the sterilization chamber 3, and the subsequent closing of the valve 7, the overheated vapour mix is fed into the sterilization chamber 3 after the valve 12 has been opened, whereby the vapour mix cools down considerably below the dew point and condenses abruptly on all accesible surfaces 2 of the objects 1 as well as on the inner walls of the sterilization chamber 3. During this abrupt condensation, an abrupt destruction of the microorganisms takes place, so that directly after the condensation, or after only a few seconds, the condensate layer can be sucked out again via the vacuum pump 5, after the valve 12 has been closed and the valve 7 has been opened, by means of further evacuation.

As mentioned above, it is an aim of the present invention to make the process even more effective, in that the overall process is accelerated and/or that the hydrogen peroxide residue, located in the sterilization chamber 3 after the condensate layer has been removed is reduced. According to FIG. 1, a heating device 14 is therefore additionally provided, with which heating elements 13 arranged in the sterilization chamber 3 are connected. Thus the inside of the sterilization chamber 3 and the surfaces 2 of the objects 1 to be sterilized can be pre-heated in a pre-determined way. An increase in the temperature of the surfaces 2 of only a few degrees Celsius results already in a considerably increased desorption rate and therefore to a considerable reduction in the amount of hydrogen peroxide residue in the sterilization chamber 3 as well as to a significantly shortened pumping-out time.

As can be seen, the sterilization chamber 3 is still connected via a valve 16 with a conduit 15 for sterile flooding gas, for example, sterile air. In order to support the reduction of hydrogen peroxide residue, this sterile air could also be heated.

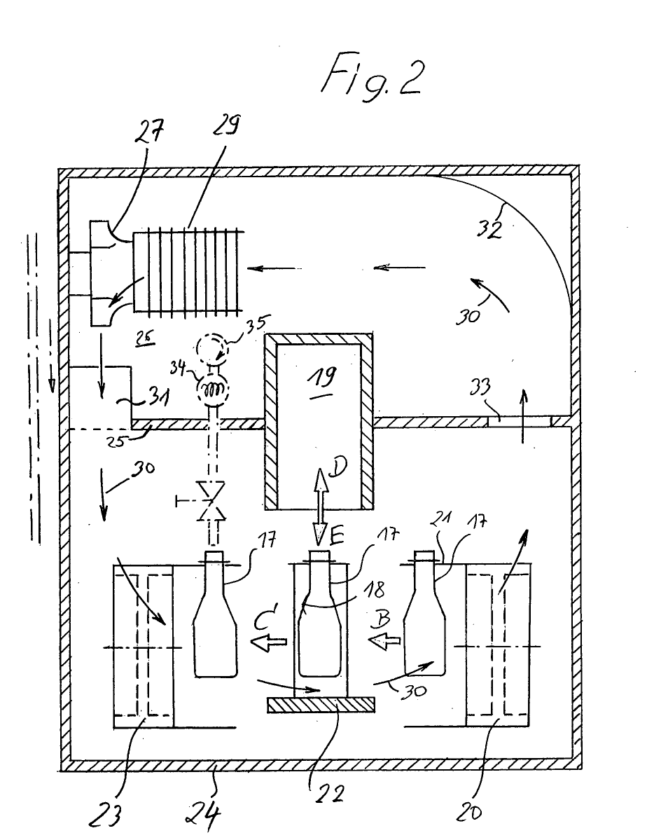

In an alternative process according to the present invention as shown in FIG. 2, it is provided that the surfaces 18 of the objects 17 to be sterilized, for example the surfaces of PET bottles, are pre-heated before the objects 17 are fed into a relevant sterilization chamber 19. This can take place by means of warm air, in particular by means of a regulated, sterile air current.

It should be noted here that the arrangement, described with the aid of FIG. 1, which serves the actual sterilization process is not shown again in the embodiment in FIG. 2.

According to FIG. 2, a feed device 20 is denoted, which is provided with holding devices 21 for the objects 17 to be sterilized. From this feed device 20, the objects 17 can be transferred in transfer direction B into a position which makes the guiding in of an object 17 or of a plurality of objects 17 into the sterilization chamber 19 possible. A lifting base 22 comprising a suitable holding device can transport the objects 17 into the sterilization chamber 19 according to the lifting direction D and hereby seal the sterilization chamber 19 closed. After sterilization, the lifting base 22 is lowered again in withdrawal direction E, whereafter the objects 17 are fed to a delivery device 23 in transfer direction C. All the mentioned devices of the entire installation can be located in one complete housing 24.

The feed device 20, the delivery device 23 as well as the inside of the sterilization chamber 19 are separated by an intermediary wall 25 from the rest of the complete housing 24, so that an antechamber 26 for a regulated, sterile air current arises. A fan 27 is located in this antechamber 26, to which fan 27 a motor (not shown) is arranged. The fan 27 is connected to a heating device 29. With the aid of the fan 27 and the heating device 29 a temperature-regulated warm air circulation 30 is generated, whereby the necessary air inlet and air outlet openings which run into the antechamber 26 are not shown. The warm air, set in motion by the fan 27, is conducted via a sterile filter 31 in the form of an absolute filter through an opening in the intermediary wall 25 in the opposite direction to delivery direction 23 and further conducted against the delivery device 20, from where the warm air then reaches the antechamber 26 again via an opening 33.

A warm air circulation 30 such as this permits the pre-heating of the surfaces 18 of the objects 17 to be sterilized in the desired way before the objects 17 are guided into the sterilization chamber 19. The sterilization chamber 19 is also, however, simultaneously heated by means of the warm air.

Denoted by a dot-dash line are an additional heat register 34 and a blowing device 35, with which, if required, insofar as a further increase in the effectiveness of the process is desired, the objects 17, shown here in the form of PET bottles, can be blown through with additional sterile hot air.

On the left side of FIG. 2 and denoted by a dot-dash line is an alternative double-walled housing 24, in which space formed by the double walls, a suitable medium, for example water steam or hot air, can circulate, which then heats the inside of the housing 24, in particular the area of the sterilization chamber 19. For this purpose, the sterilization chamber 19 itself can also be double-walled (not shown).

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

1. A process for sterilizing objects in a sterilization chamber in which a vacuum prevails, in which sterilization chamber a vapour mix consisting of water steam and hydrogen peroxide steam is fed without the use of carrier gas, the said vapour mix being deposited in the form of a condensate layer abruptly on the surfaces of the objects to be sterilized and on the surfaces of the sterilization chamber, whereafter the condensate layer is subsequently sucked out by means of further evacuation of the sterilization chamber, wherein at least one of the surfaces of the objects to be sterilized and the sterilization chamber are pre-heated to a pre-determined temperature.

2. A process according to claim 1, wherein the surfaces are pre-heated by means of installed heating devices.

3. A process according to claim 1, wherein the surfaces are pre-heated with warm air.

4. A process according to claim 3, wherein a regulated sterile air current supplies the warm air.

5. A process according to claim 1, wherein the entire sterilization chamber is pre-heated.

6. A process according to claim 1, wherein individual areas of the sterilization chamber are intentionally pre-heated.

7. A process according to claim 1, wherein the objects are pre-heated before they are guided into the sterilization chamber.

8. A process according to claim 1, wherein the objects are subject to a rapid-acting hot air current after the condensate layer has been sucked out.
